(19) United States
(12) Patent Application Publication
(10) Pub. No.: US 2020/0279619 A1
(43) Pub. Date: Sep. 3, 2020

(54) TAILORED ADVICE FOR COPD PATIENTS

(71) Applicant: KONINKLIJKE PHILIPS N.V., EINDHOVEN (NL)

(72) Inventors: STEFAN WINTER, AACHEN (DE); VIOLETA MOOKHOEK, EINDHOVEN (NL)

(21) Appl. No.: 16/799,891

(22) Filed: Feb. 25, 2020

Related U.S. Application Data
(60) Provisional application No. 62/812,401, filed on Mar. 1, 2019.

(51) Int. Cl. G16H 10/20; A61B 5/00; G16H 40/67; G10L 15/22; G10L 15/02

(52) U.S. Cl. CPC G16H 10/20; A61B 5/4803; G10L 15/02; G10L 15/22; G16H 40/67

(57) ABSTRACT

Telehealth was introduced as a promising service to support integrated care in management of chronic obstructive pulmonary disease (COPD). Implementation of telehealth is geared towards improving self-management and providing remote support for continuous disease management. However, it is currently not clear where telehealth services fit into existing pathways for COPD disease management. This exploratory study aims to identify the practices of clinical stakeholders along a Greek clinical care pathway for COPD, detail the challenges of those practices, and to identify where telehealth solutions may be implemented to reduce clinical resource utilization.

### CROSS-REFERENCE TO RELATED APPLICATIONS

This patent application claims the priority benefit under 35 U.S.C. § 119(e) of U.S. Provisional Application No. 62/812,401, filed on Mar. 1, 2019, the contents of which are herein incorporated by reference.

### BACKGROUND OF THE INVENTION

### 1. Field of the Invention

The present invention pertains to a method and apparatus for monitoring and controlling a pressure support device, and, in particular, a program that is tailored to provide a patient with information relevant for the specific needs of that patient.

### 2. Description of the Related Art

Managing diseases like COPD can be quite complex and physicians face the challenge that they have to focus their precious time on few educational topics when they see the patient. Even then, they cannot be sure that the patient is following the advice. While there are solutions out there that offer the patient to review the information and learn even more at home they lack personalization taking into account the already given advice by the physician, the environment and the personal situation of the patient at home.

### SUMMARY OF THE INVENTION

The proposed solution automatically composes a tailored program with information to be reviewed provided by the physician and adding new information that is relevant for the patient and context specific.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of the overall system of the present invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

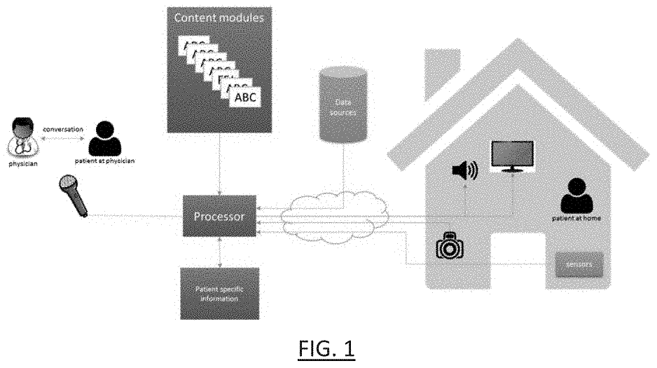

The proposed solution, which is illustrated schematically in FIG. 1, includes a microphone situated in the location where the physician instructs the patient to record the conversation. The recorded conversation is transferred to a processor that converts it to written text and then analyses its content, for example, by pre-defined keywords, extracts patient-specific information such as amount of medication and structures the extracted content by clustering it to identify addressed topics in the next step.

The content clusters are compared to pre-defined content modules that cover the various information items a patient needs to learn. This can be achieved, for example, by comparing key words and their synonyms that reflect content that needs to be addressed in the modules. For each module that is covered by one of the content clusters, the system remembers that it has been covered by the physician along with the patient-specific information.

The solution further contains one or more information sharing interfaces at the patient's home such as loudspeakers or screens. Once at home the system offers the patient the information covered by the physician for review. Each module also contains tests such as multiple-choice tests that the patient needs to answer to check his understanding. Those modules with lower test results are repeated more often than others.

In addition, the system can offer new information based on content modules that have not been covered by the physician but that are relevant for the patient. To this end, the content modules are marked as either mandatory because they are relevant for all patients (within a disease-specific sub-group) or as optional and assigned attributes that indicate context-specific relevance.

For example, a module advising the patient not to leave the accommodation if it is hot outside could be marked with the attribute ‘outside temperature larger 28° C.’. Another module could have the attribute ‘fireplace turned on’ and offers specific information for owners of a fireplace that is in use. To offer such relevant information at the right time the system is connected to data sources that provide context information such as current or forecasted weather, location of accommodation, activity trackers, . . . . These data sources can be added and removed dynamically based on their availability.

Collecting the information shared by the physician can alternatively or in combination also happen by text that the physician enters in a system dedicated for the described system or another system he uses like the electronic medical record (EMR). One specific source of information would be extracted from a multitude of pictures taken at the patient's home. Those pictures are analyzed by image- and object-recognition software to detect items that appear in the content modules' attributes such as a fireplace.

Instead of at home reviewing and getting new information can also happen in other locations such as the hospital. In another embodiment informal care givers can be included by receiving the information that the patient needs to review and share it with patient.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A system for providing information to a patient comprising:
an input device adapted to receive first information from a caregiver;
an information processor adapted (a) extract key identifiers from the first information, and (b) select a content module from a plurality of content modules based on the key identifier; and
an output device adapted to provide the content module to the patient selected by the information processor.

2. The system of claim 1, wherein the input device is a microphone.

3. The system of claim 1, wherein the content module includes a questionnaire to be answered by the patient.
